(19) United States
(12) Patent Application Publication
(10) Pub. No.: US 2018/0036050 A1
(43) Pub. Date: Feb. 8, 2018

(54) BONE PLATE

(71) Applicant: Kaushal Kant Mishra, New Delhi (IN)

(72) Inventor: Kaushal Kant Mishra, New Delhi (IN)

(21) Appl. No.: 15/694,896

(22) PCT Filed: Mar. 3, 2016
(86) PCT No.: PCT/IB2016/051195

§ 371 Date: Sep. 4, 2017

(30) Foreign Application Priority Data
Mar. 5, 2015 (IN) 628/DEL/2015

(51) Int. Cl. A61B 17/80; A61F 2/28

(52) U.S. Cl. CPC A61B 17/8052; A61F 2/28; A61F 2002/30136

(57) ABSTRACT

Disclosed is a bone plate (10) for a bone having fracture. The bone plate (10) is used with fastening means/screws (24) for fixing the bone plate (10) with the bone (40). The present bone plate (10) is also appropriate for periprosthetic fracture (46) as the fixing of the bone plate (10) involves screwing/fixing of the bone plate (10) through cortex portion (42) of the bone. The present bone plate (10) is a multipurpose bone plate (10) having wavy body that enables treatment of bone (40) having fracture (46) at any part of the body for all types of long bone. Further, the bone plate (10) is configured with ridge (34) and multiple elevations (32) at the bone facing surface (30) which results in minimal bone (40) to bone plate (10) contact area allowing appropriate circulation of blood around the bone interface for appropriate healing of the fracture.

### FIELD OF THE INVENTION

The present invention generally relates to bone plates for bone having fracture. More particularly, the present invention relates to an improved bone plate that can be used in all type of long bone fractures including periprosthetic fractures.

### BACKGROUND OF THE INVENTION

The treatments of bone fractures have there own fixation challenges and affects. There are different types of fractures associated with bones, depending on the location/part of impact on the bone. The fractures at different part of the bone needs to be fixed with different bone plates having different shapes and features. Thus, there is no single bone plate that can suffice the need of implementation of a single bone plate for bone having different fractures at different parts of the body.

Further, there is always a possibility of new fractures associated with previous fractures. In such a scenario, one of the pre-fixed bone plate and nail inserted into a bone for addressing the earlier bone fracture creates quandary during the periprosthetic (post-treatment) treatment of the bone fracture. More specifically, once a bone fracture is addressed in a traditional way of nailing arrangement (that extends through the medullary portion of the bone), there are multiple limitations in terms of addressing any new fracture in the same bone. Traditionally there have been some solutions provided which would only partially address the situations and the same are not medically sound and have many post operative challenges.

Furthermore, the existing bone plates have limitations of having larger contact area over the bone which is essential for fixation of such bone plates with the bone. Therefore, the larger contact area results in limited blood circulation around the bone and specifically between the tissues and the bone which eventually results in inappropriate healing of the fracture.

Accordingly, there exists a need for a multipurpose single bone plate having minimal bone to bone plate interface that can be used in the treatment of almost all types of bone fractures including periprosthetic fracture. There also lies a need of bone plate that is appropriate for treatment of periprosthetic fractures.

Further, there exists a need of bone plate that do not craft setback to blood circulation due to larger bone facing contact area of the bone plate without compromising on the stability of fixation of the bone plate to the bone.

### SUMMARY OF THE INVENTION

The present invention provides a bone plate for a bone having fracture. In view of the foregoing disadvantages inherent in the prior-art, the general purpose of the present invention is to provide a multi purpose bone plate that is configured to include all advantages of the prior art and to overcome the drawbacks inherent in the prior art and offering added advantages.

The present bone plate is an extra-medullary, transcortical, minimal contact, internal fixation plating device. Accordingly, the present invention provides a bone plate for a bone having fracture that can be fixed internally in to a bone having fracture. The bone plate having an elongated wavy body extending longitudinally from one end to another end. The wavy body having at least a crest portion and at least a trough portion. Further, the bone plate involves fixation of the bone plate with the bone using screws/fastening means. The screws/fastening means are penetrated through the cortex portion of the bone (transcortical) avoiding penetration through medullary section of the bone (extra-medullary).

Furthermore, the bone plate having extended portion on the bone facing surface allows minimal bone to bone plate contact area thereby providing better blood circulation around the bone plate and bone interface ebabling appropriate healing of the bone fracture.

The bone plate comprising: a first surface exposed to be in contact with a tissue; a second surface opposite to the first surface exposed to be in contact with the bone; at least a first slot configured at the at least a crest portion and the at least a trough portion of the body, the at least a first slot extending from the first surface of said bone plate to the second surface of said bone plate; at least a second slot configured longitudinally along the central axis of said bone plate, the at least a second slot extending from the first surface of the bone plate to the second surface of said bone plate; wherein the at least a first slot and the at least a second slot having at least a first edge at the first surface of said bone plate and at least a second edge at the second surface of said bone plate; at least an elevation configured at the at least a second edge of the at least a first slot; and a ridge provided at the second surface, the ridge extending longitudinally along the central axis of said bone plate;

### BRIEF DESCRIPTION OF THE DRAWINGS

The advantages and features of the present invention will become better understood with reference to the following detailed description and claims taken in conjunction with the accompanying drawing, in which:

FIG. 1 illustrates top perspective view indicating the first surface of the bone plate according to an examplarery embodiment of the present invention;

FIG. 1a illustrates close-up view indicating the first surface of the bone plate of FIG. 1 according to an examplarery embodiment of the present invention;

FIG. 2 illustrates bottom perspective view indicating the second surface of the bone plate according to an examplarery embodiment of the present invention.

FIG. 2a illustrates close-up view indicating the second surface of the bone plate of FIG. 2 according to an examplarery embodiment of the present invention;

FIG. 3 illustrates top view of the bone plate according to an examplarery embodiment of the present invention;

FIG. 4 illustrates bottom view of the bone plate according to an examplarery embodiment of the present invention;

FIG. 5 illustrates lateral side view of the bone plate according to an examplarery embodiment of the present invention;

FIG. 6 illustrates front view of the bone plate according to an examplarery embodiment of the present invention;

FIG. 7 illustrates perspective view of an embodiment of the bone plate fixed/placed on a bone having fracture in an embodiment according to an examplarery embodiment of the present invention; and

FIG. 8 illustrates front cross-sectional view of the bone plate exemplifying placement of fastening means that avoid penetration of the fastening means through the medullary section of the bone according to an examplarery embodiment of the present invention.

### DETAILED DESCRIPTION OF THE INVENTION

The exemplary embodiments described herein detail for illustrative purposes are subject to many variations in structure and design. It should be emphasized, however, that the present invention is not limited to a particular bone plate as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The use of terms “including,” “comprising,” or “having” and variations thereof herein are meant to encompass the items listed thereafter and equivalents thereof as well as additional items. Further, the terms, “a” and “an” herein do not denote a limitation of quantity, but rather denote the presence of at least one of the referenced item.

The present bone plate is an evolution over the existing bone plates wherein bone plates were subjected to be used for specific types of fractures at specific location/part of the bone in the body. The present bone plate bears all the features of the existing bone plate along with additional features that resolve the shortcomings associated with the existing bone plates.

The present bone plate can be used in all types of long bone fractures and at any part of the long bone. Further, the present invention is appropriate for addressing bone fractures that have previous history of fracture(s) or any on-going treatment of fracture(s) such as treating the fracture (46) by nailing through the medullary section of the bone.

The present bone plate involve fixation of the bone plate with the bone by screwing the bone plate through the cortex portion of the bone without affecting the medullary section of the bone. The fixation of the bone plate is done by screwing the bone plate through the cortex portion of the bone and avoiding penetration through the medullary section of the bone which allows treatment of the bone having a subsequent fracture wherein a nail is already inserted in the medullary section of the bone for treating a previous fracture associated with the bone. The bone plate further has a bone facing surface and a surface opposite to the bone facing surface.

The present bone plate has an extended ridge and elevation at the bone facing surface that allows mounting the bone plate on the bone with a minimal bone to bone plate contact area. Further, the present bone plate involves three point locking system for better stability and strength of fixation of the bone plate with the bone. The extended ridge and elevation allows better blood circulation around the bone plate and bone interface enabling appropriate healing of the bone fracture.

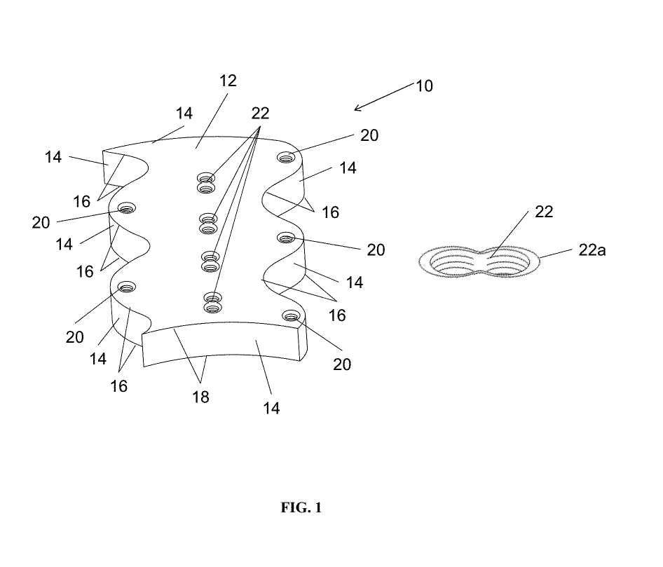

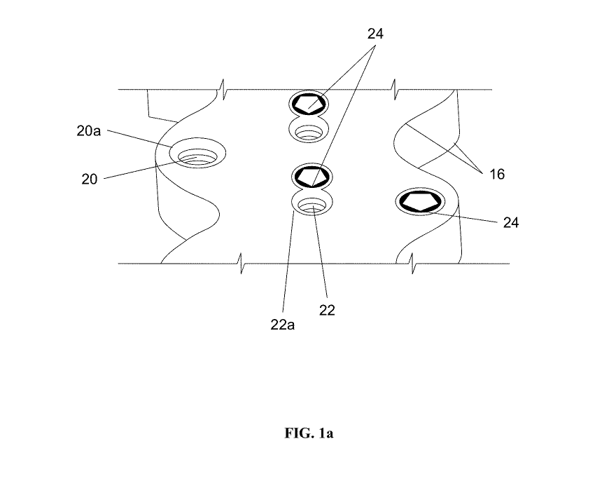

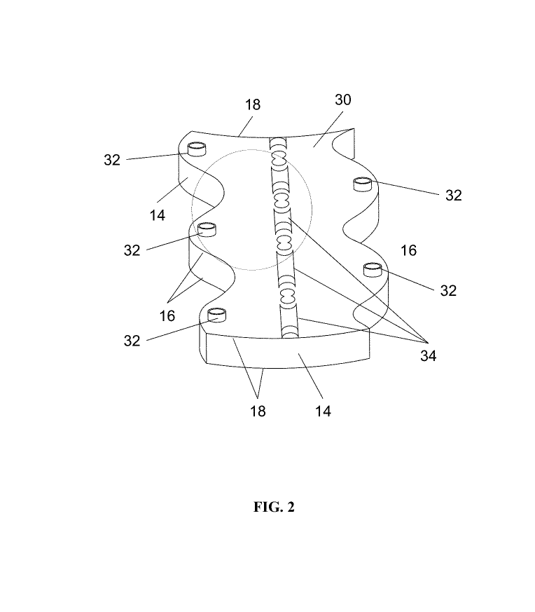

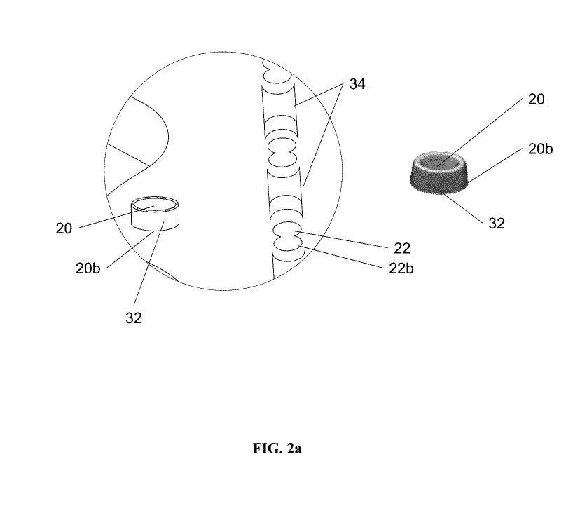

Referring to FIG. 1, FIG. 1a, FIG. 2 and FIG. 2a, illustrated are perspective views of the bone plate (10) indicating the bone facing surface (herein after referred to as second surface (30)) referred in FIG. 2 and FIG. 2a and the surface opposite to the bone facing surface (herein after referred to as first surface (12)) of the bone plate (10) having plurality of slots referred in FIG. 1 and FIG. 1a. The bone plate (10) having a wavy body as a result qualifying a plurality of crest portions (36) and plurality of trough portions (38). The slots present at the bone plate (10) are combinational holes (herein after referred to as combi-hole) meant for receiving fastening means for fixing the bone plate (10) with the bone having fracture. The combinational holes are the holes that enable conventional screwing technique as well as locked screwing technique depending on the surgical requirement. Further, the plurality of combinational holes are divided into two sets of slots, first set of slots (20) being present at one of the crest portion (36) and trough portion (38) of the bone plate (10) and second set of slots (22) being present along the longitudinal axis of the bone plate.

There are plurality of edges formed at the junction of the periphery of the first set of slots (20) and the second set of slots (22) with one of the first surface (12) and the second surface (30). First set of edges formed at the first surface (12) referred to the edges that form at the junction between the first surface (12) and periphery of first set of slots (20) and second set of slots. Second set of edges formed at the second surface (30) referred to the edges that form at the junction between the second surface (30) and periphery of first set of slots (20) and second set of slots. Further, plurality of elevations (32) configured at the second set of edges at the second surface (30) of the first set of slots. The elevation (32) at the second set of edges at the second surface (30) of the first set of slots (20) is configured to provide minimal bone plate (10) contact with the bone for better circulation of blood around the bone and to provide better stability of fixation of the bone plate (10) with the bone.

The fastening means for fixing the bone plate (10) of the present invention involve two variants of screws, one having semi-spherical ball shaped head for tightening the bone plate (10) so as to push the bone plate (10) towards the bone and other having patches on the head for locking the head with the bone plate (10) by screwing. Further, the screws (24) inserted through the two adjacent first set of slots (20) and one diagonally opposite second set of slot provide three point locking system for proper stability and strength of fixation of the bone plate (10) with the bone.

The bone plate (10) having plurality of side walls (14) that connect the first surface (12) and the second surface (30) wherein the second surface (30) is the bone facing surface and the first surface (12) is the surface opposite to the bone facing surface. Further, plurality of edges (16, 18) are formed at the junction of plurality of side walls (14) and between a side wall (14) and one of first surface (12) and second surface (30). The first set of edges is longitudinal edges (16) which contribute to the length along the longitudinal axis of the bone plate (10) and the second set of edges is transverse edges (18) which contribute to the Plano-concave shape of the bone plate (10) along the transverse axis. Furthermore, the edges formed at the junction between longitudinal side walls (14) and the first surface (12) contribute to the convex shape of the first surface (12) and the edges formed at the junction between longitudinal side walls (14) and the second surface (30) contributes to the concave shape of the second surface (30). However, it should be evident to a person skilled in the art that the bone plate (10) of the present invention can also be configured without the plurality edges being formed between plurality of side walls and the first surface (12) and the second surface (30). More specifically the first surface and the second surface may have surface edges to transition to the side walls.

A ridge (34) is configured at the second surface (30) of the bone plate (10) which is extended along the longitudinal axis of the bone plate (10). Further, the ridge (34) present at the second surface (30) is not continuous and is absent for a reasonable distance from the second set of edges (22b) of the second set of slots (22). The ridge (34) is configured to provide minimal bone plate (10) contact for better circulation of blood around the bone plate (10).

The plurality of elevations (32) at the second edge (20b) of the first set of slots (20) and the ridge (34) configured at the second surface (30) has the same thickness. The point of contact of the bone plate (10) with the bone is the elevations (32) and the ridge (34) configured at the second surface (30) of the bone plate (10).

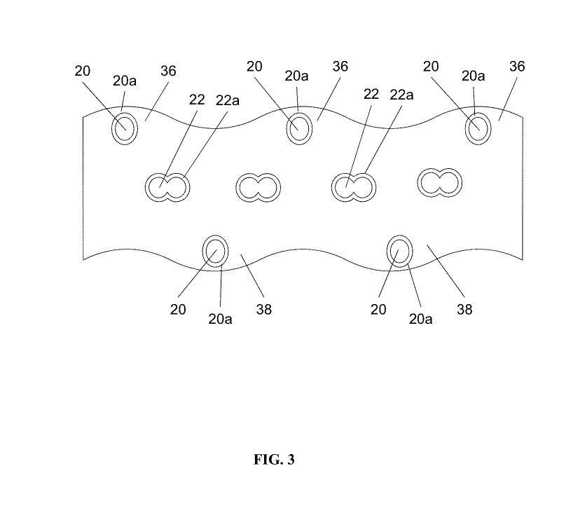

Referring to FIG. 3 to FIG. 6, illustrated are different views of the bone plate (10) of the present invention. Specifically, FIG. 3 of the present invention refer to the top view of the bone plate (10) wherein the first surface (12) of the bone plate (10) is visible along with the plurality of the first set of slots (20) and the second set of slots (22). The first set of slots (20) being present at the crest (36) portion and the trough (38) portion of the bone plate (10). The second set of slots (22) being present along the longitudinal axis of the bone plate (10).

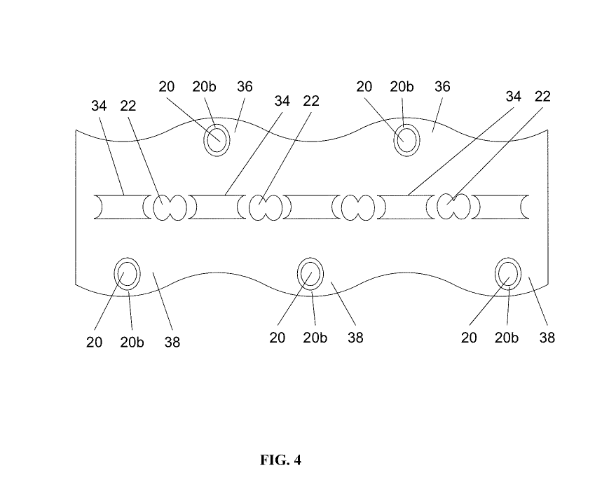

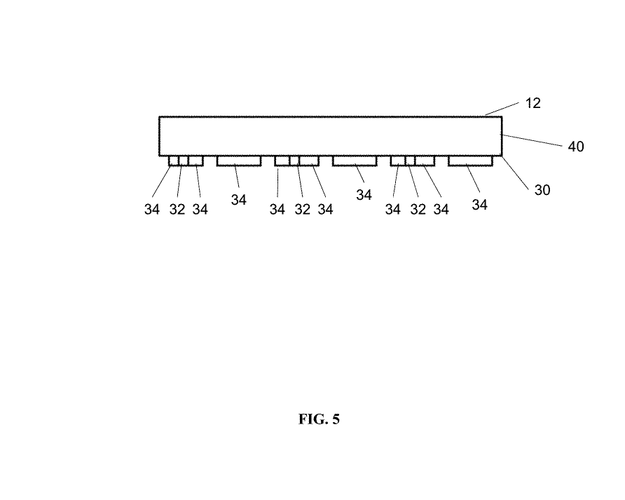

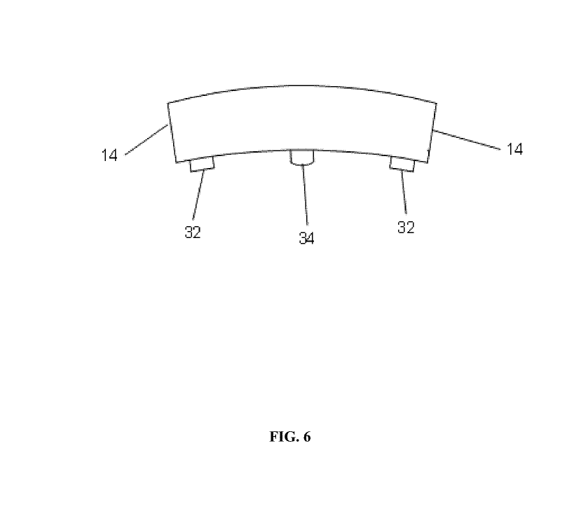

FIG. 4 of the present invention refers to the bottom view of the bone plate (10) wherein the second surface (30) of the bone plate (10) is visible wherein plurality of the first set of slots (20) and the second set of slots (22) are configured. The first set of slots (20) being present at the crest (36) portion and the trough (38) portion of the bone plate. The second set of slots (22) being present at the longitudinal axis of the bone plate (10). Further, ridge (34) is configured along the longitudinal axis of the bone plate (10) wherein the ridge (34) is not continuous and is absent for a reasonable distance from the second set of edges of the second set of slots (22) at the second surface (30). FIG. 5 of the present invention refers to the side view of the bone plate (10) wherein the thickness of the ridge (34) is visible along with the ridge (34) and the elevation (32) configured at the second edge surface (30) of the bone plate (10). FIG. 6 of the present invention refers to the front view of the bone plate (10) indicating the thickness of the ridge (34) and the elevation (32) configured at the second surface (30). The thickness of the elevations (32) and ridge (34) represent the extended portion from the second set of edges (20b, 22b) at the second surface (30).

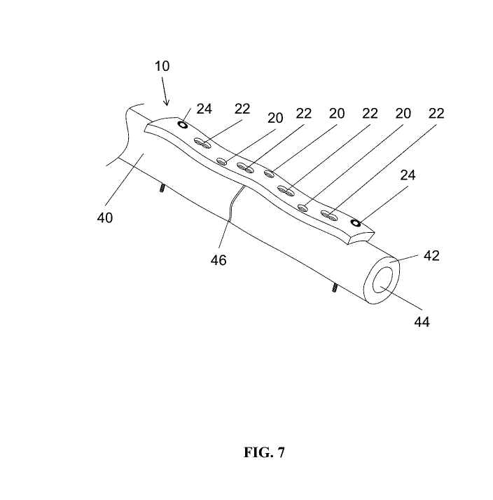

Referring to FIG. 7, illustrated is the top perspective view of the bone plate (10) placed over a bone (40) having fracture (46) in an exemplarery embodiment of the present invention. The bone plate (10) having plurality of crest (36) portions and trough (38) portions those contribute to the wavy shape of the bone plate (10) body. Further, fastening means (24) of the bone plate (10) are penetrated through the bone (40) for fixing the bone plate (10) with the bone (40).

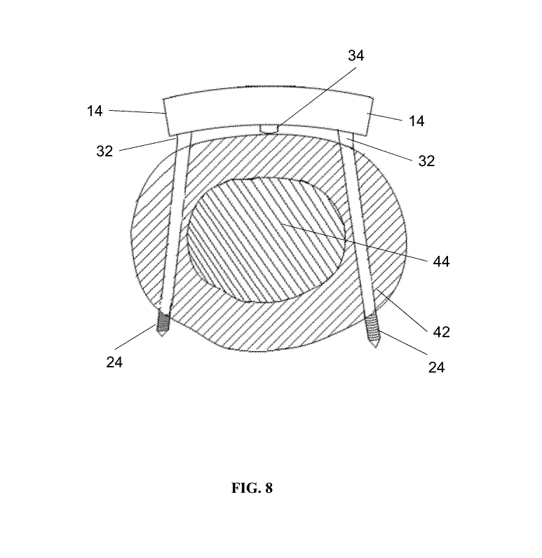

Referring to FIG. 8, illustrated is the front cross-sectional view of the bone plate (10) fixed on a bone (40) having fracture (46) in an embodiment. The figure illustrates a set of screws (24) being inserted at certain insertion angles to avoid penetration through the medullary section (44) of the bone (40). The set of screws (24) are inserted and fixed through the cortex (42) portion of the bone (40). Further, the points of the contact of the bone plate (10) with the bone are plurality of elevations (32) configured at the second surface (30) and the ridge (34) configured along the longitudinal axis of the second surface (30).

The device/system of the bone plate (10) described and illustrated in the exemplary embodiment as discrete without departing from the scope of the present invention. Other examples of changes, substitutions, and alterations ascertainable by one skilled in the art, upon studying the exemplary embodiment disclosed herein, may be made without departing from the spirit and scope of the present invention. For instance, the present bone plate may have smooth surface instead of the plurality of side walls (14) and plurality of edges (16, 18).

The advantage of the present bone plate (10) is an Extra-medullary, transcortical, minimal contact, internal fixation plating device. Accordingly, the present invention provides a bone plate (10) for a bone (40) having fracture that can be fixed internally into a bone (40) having fracture (46). The bone plate (10) involves fixation of the bone plate (10) with the bone (40) using screws/fastening means (24). The screws/fastening (24) means are penetrated through the cortex (42) portion of the bone (40) avoiding penetration through medullary (44) section of the bone (40). Further, the bone plate (10) having extended portion on the bone facing surface that allows minimal bone to bone plate (10) contact area providing better blood circulation around the bone plate and bone interface, such bone plate enables appropriate healing of bone fracture.

It should be noted that reference throughout this specification to one of features, advantages and similar language does not imply that all of the features and advantages should be or are in any single embodiment. Rather, language referring to the features and advantages may be understood to mean that one of a specific feature, advantage, and characteristic described in connection with an embodiment may be included in at least one embodiment of the present invention. Thus, discussions of the features and advantages, and similar language, throughout this specification may, but do not necessarily, refer to the same embodiment.

I claim:

1. A bone plate (10) for a bone, wherein said bone plate (10) has at least a crest (36) portion and at least a trough (38) portion, said bone plate (10) comprising:
a first surface (12) exposed to be in contact with a tissue;
a second surface (30) opposite to the first surface (12) exposed to be in contact with the bone;
at least a first slot (20) configured at the at least a crest (36) portion and the at least a trough (38) portion of said bone plate (10), the at least a first slot (20) extending from the first surface (12) of said bone plate (10) to the second surface (30) of said bone plate;
at least a second slot (22) configured longitudinally along the central axis of said bone plate, the at least a second slot (22) extending from the first surface (12) of said bone plate (10) to the second surface (30) of said bone plate;
wherein the at least a first slot (20) and the at least a second slot (22) having at least a first edge (20a, 22a) at the first surface (12) of said bone plate (10) and at least a second edge (20b, 22b) at the second surface (30) of said bone plate;
at least an elevation (32) configured at the at least a second edge (20b, 22b) of the at least a first slot; and
a ridge (34) provided at the second surface (30), the ridge (34) extending longitudinally along the central axis of said bone plate;

2. The bone plate (10) as claimed in claim 1, wherein said first surface (12) is convex shaped.

3. The bone plate (10) as claimed in claim 2, wherein a first set of edges (16) formed at the junction of said first surface (12) and at least a side wall (14) contributes to the convex shape of said first surface (12).

4. The bone plate (10) as claimed in claim 1, wherein said second surface (30) is concave shaped.

5. The bone plate (10) as claimed in claim 3, wherein said first set of edges (16) formed at the junction of said second surface (30) and at least a side wall (14) contributes to the concave shape of said second surface (30).

6. The bone plate (10) as claimed in claim 1, wherein said second set of edges (18) contribute to the Plano-concave shape of said bone plate (10).

7. The bone plate (10) as claimed in claim 1, wherein said at least a first slot (20) and said at least a second slot (22) is capable for receiving at least a fastening means (24) for fixing said bone plate (10) with said bone.

8. The bone plate (10) as claimed in claim 7, wherein said at least a first slot (20) and said at least a second slot (22) is capable of receiving said at least a fastening means (24) at a variable insertion angle.

9. The bone plate (10) as claimed in claim 8, wherein said insertion angle is chosen in a way to avoid penetration through medullary (44) section of the bone.

10. The bone plate (10) as claimed in claim 1, wherein said at least a second slot (22) is combi-hole effective for conventional screwing technique and locked screwing technique.

11. The bone plate (10) as claimed in claim 1, wherein two said at least a first slot (20) provided at said crest (36) portions and said at least a first slot (20) provided at said trough (38) portion contributes to configure said three point locking system.

12. The bone plate (10) as claimed in claim 1, wherein two said at least a first slot (20) provided at said trough (38) portions and said at least a first slot (20) provided at said crest (36) portion contributes to configure said three point locking system.

13. The bone plate (10) as claimed in claim 1, wherein said at least a first edge (20a, 22a) at said first surface (12) of said at least a first slot (20) and at least a second slot (22) is formed at a junction of said first surface (12) and periphery of said at least a first slot (20) and said at least a second slot (22) at said first surface (12).

14. The bone plate (10) as claimed in claim 1, wherein said at least a second edge (20b, 22b) at said second surface (30) of said at least a first slot (20) and at least a second slot (22) is formed at a junction of said second surface (30) and periphery of said at least a first slot (20) and at least a second slot (22) at said second surface (30).

15. The bone plate (10) as claimed in claim 14, wherein said elevation (32) is configured at said at least a second edge (20b, 22b) of said at least first slot (20) for providing a minimal bone to bone plate (10) contact.

16. The bone plate (10) as claimed in claim 15, wherein said elevation (32) at said least a second edge (20b, 22b) of said at least first slot (20) has a thickness for allowing minimal bone to bone plate (10) contact.

17. The bone plate (10) as claimed in claim 1, wherein said ridge (34) is provided at said second surface (30) for providing minimal bone to bone plate (10) contact.

18. The bone plate (10) as claimed in claim 17, wherein said ridge (34) at said second surface (30) has a thickness for allowing minimal bone to bone plate (10) contact.

19. The bone plate (10) as claimed in claim 1, wherein said elevation (32) and said ridge (34) has same thickness.
